(19) United States
(12) Patent Application Publication
(10) Pub. No.: US 2003/0186256 A1
(43) Pub. Date: Oct. 2, 2003

(54) Method for carrying out the parallel sequencing of a nucleic acid mixture on a surface

(76) Inventor: Achim Fischer, Heidelberg (DE)

Correspondence Address: BIRCH STEWART KOLASCH & BIRCH, PO BOX 747, FALLS CHURCH, VA, 22040-0747, US

(21) Appl. No.: 10/168,557

(22) PCT Filed: Dec. 22, 2000
(86) PCT No.: PCT/EP00/13157

(30) Foreign Application Priority Data
Dec. 23, 1999 (DE) 19962893.9
Oct. 18, 2000 (DE) 10051564.9

(51) Int. Cl. C12Q 1/68; C12P 19/34

(52) U.S. Cl. 435/6; 435/91.2

(57) ABSTRACT

The invention relates to a method for sequencing in parallel at least two different nucleic acids present in a nucleic acid mixture, characterized in that

(a) a surface is provided, which surface possesses islands of nucleic acids of in each case the same type, i.e. tertiary nucleic acids;

(b) counterstrands of the tertiary nucleic acids, i.e. TNCs, are provided;

(c) the TNCs are extended by one nucleotide, with

the nucleotide at the 2′-OH position or at the 3′-OH position carrying a protecting group which prevents further extension,

the nucleotide carrying a molecular group which enables the nucleotide to be identified;

(d) the incorporated nucleotide is identified;

(e) the protecting group is removed and the molecular group of the incorporated nucleotide, which is used for identification, is removed or altered, and

(f) step (c) and subsequent steps are repeated until the desired sequence information has been obtained.

The invention relates to a method for the solid phase-supported sequencing in parallel of at least two different nucleic acids present in a nucleic acid mixture.

Sequence analysis of nucleic acids is an important method in biological analysis. The method determines the precise sequence of the bases in the DNA or RNA molecules of interest. Knowledge of this base sequence makes it possible, for example, to identify particular genes or transcripts, that is the messenger RNA molecules pertaining to these genes, to uncover mutations or polymorphisms, or else to identify organisms or viruses which can be recognized unambiguously with the aid of particular nucleic acid molecules. Nucleic acids are customarily sequenced using the chain termination method (Sanger et al. (1977) PNAS 74, 5463-5467). For this, a single strand is enzymically converted into the double strand by a “primer”, which is hybridized to said single strand and which is as a rule a synthetic oligonucleotide, being extended by means of adding DNA polymerase and nucleotide building blocks. The addition of a small amount of termination nucleotide building blocks, which, after having been incorporated into the growing strand, do not permit any further extension, leads to the accumulation of constituent strands possessing known ends which are specified by the respective termination nucleotide. The mixture of strands of differing length obtained in this way is fractionated according to size by gel electrophoresis. The nucleotide sequence of the unknown strand can be derived from the band patterns which are produced. A major disadvantage of said method is the instrumental input which is required, which input restricts the throughput of reactions which can be achieved. Assuming that four different fluorophoree-labeled termination nucleotides are used, each sequencing reaction requires at least one line on a flat gel, or at least one capillary when capillary electrophoresis is employed. In what are at present the most modern automated sequencers which are commercially available, the input arising from this restricts the number of sequencings which can be processed in parallel to a maximum of 96. Another disadvantage consists in the restriction in the reading length, that is the number of bases which can be correctly identified per sequencing, due to the resolution of the gel system. While an alternative method of sequencing, i.e. determining the sequence by means of mass spectrometry, is faster, and therefore enables more samples to be processed in the same amount of time, this method is, on the other hand, restricted to relatively small DNA molecules (for example 40-50 bases). In another sequencing technique, i.e. sequencing by hybridization (SBH; cf. Drmanac et al., Science 260 (1993), 1649-1652), base sequences are identified by the specific hybridization of unknown samples with known oligonucleotides. For this purpose, said known oligonucleotides are attached to a support in a complex arrangement, a hybridization with the labeled nucleic acid to be sequenced is performed, and the hybridizing oligonucleotides are identified. The sequence of the unknown nucleic acid can then be determined from the information with regard to which oligonucleotides have hybridized with the unknown nucleic acid and the sequence of the oligonucleotides. A disadvantage of the SBH method is the fact that the optimum hybridizing conditions for oligonucleotides cannot be predicted precisely and it is accordingly not possible to design any large aggregate of oligonucleotides which, on the one hand, contain all the possible sequence variations for their given length and which, on the other hand, require precisely the same hybridization conditions. As a consequence, errors occur in the sequence determination as a result of nonspecific hybridization. In addition, it is not possible to use the SBH method for repetitive regions in nucleic acids which are to be sequenced.

In addition to the analysis of the strength with which known genes are expressed, as can be achieved by dot blot hybridization, northern hybridization and quantitative PCR, methods are also known which enable unknown genes, which are expressed differentially between different biological samples, to be identified de novo.

A strategy of this nature for analyzing expression consists in quantifying discrete sequence units. These sequence units can comprise what are termed ESTs (expressed sequence tags). If sufficient numbers of clones obtained from cDNA libraries derived from samples which are to be compared with each other are sequenced, it is possible to recognize and count sequences which are in each case identical and to compare the resulting relative frequencies of these sequences in the different samples (cf. Lee et al., Proc. Natl. Acad. Sci. U.S.A. 92 (1995), 8303-8307). Different relative frequencies of a particular sequence indicate differential expression of the corresponding transcripts. However, the described method is very elaborate since it is necessary to sequence many thousand clones even for quantifying the more frequent transcripts. On the other hand, only a short sequence segment of approx. 13-20 base pairs in length is as a rule required for unambiguously identifying a transcript. The method of “serial analysis of gene expression” (SAGE) makes use of this fact (Velculescu et al., Science 270 (1995), 484-487). In this method, short sequence segments (tags) are concatenated and cloned and the resulting clones are sequenced. In this way, it is possible to determine about 20 tags using a single sequencing reaction. However, this technique is still not very efficient since very many conventional sequencing reactions have to be carried out and analyzed even for quantifying the more frequent transcripts. Because of the high input, it is only with very great difficulty that it is possible to use SAGE to reliably quantify rare transcripts.

According to U.S. Pat. No. 5,695,934, another method for sequencing tags comprises coating small spheres with the nucleic acid to be sequenced in such a way that each sphere receives a large number of molecules of only one nucleic acid species. The method of “stepwise ligation and cleavage” is then used for the sequencing; in this method, the nucleic acid to be sequenced is disassembled base by base, and its sequence determined at the same time, using a type IIS restriction enzyme and proceeding from an artificial linker. In order for it to be possible to observe and record the sequencing process, the spheres which are employed are introduced into a shallow cuvette, which is only a little taller than the sphere diameter, in order to enable a single layer to be formed. In addition, the spheres must be packed as densely as possible in the cuvette so as to ensure that there is no change in the arrangement of the spheres during the sequencing process, either as a result of the necessary exchange of reaction solutions or as a result of the appliance being jolted. Although it is possible to carry out many sequencing reactions in a small space in this way, the arrangement in a very narrow cuvette (a few micrometers in height) suffers from substantial disadvantages since it is difficult to fill the cuvette uniformly. Another disadvantage is the high input of apparatus which the method requires. For example, it is necessary to carry out the method using high pressures in order to enable the necessary reaction solutions to be exchanged efficiently despite the small size of the cuvette. Yet another disadvantage is that it is easy for the cuvette to become blocked, something which is likewise favored by the necessarily small dimensions of the cuvette.

The known methods for analyzing nucleic acids suffer from one or more of the following disadvantages:

They only enable to a very restricted extent individual sequencing reactions to be carried out in parallel.

They require relatively large quantities of the nucleic acid whose sequence is to be predetermined.

They are only suitable for determining the sequences of short sequence segments and require a high input of apparatus.

It is the object of the invention to provide a method which overcomes the disadvantages of the prior art.

The object according to the invention is achieved by means of a method for sequencing in parallel at least two different nucleic acids present in a nucleic acid mixture, where

(a) a surface, possessing islands of nucleic acids of in each case the same type, i.e. tertiary nucleic acids, is provided;

(b) counterstrands of the tertiary nucleic acids, i.e. TNCs, are provided;

(c) the TNCs are extended by one nucleotide, with

the nucleotide at the 2′-OH position or at the 3′-OH position carrying a protecting group which prevents further extension,

the nucleotide carrying a molecular group which enables the nucleotide to be identified;

(d) the incorporated nucleotide is identified;

(e) the protecting group is removed and the molecular group of the incorporated nucleotide, which is used for identification, is removed or altered, and

(f) step (c) and subsequent steps are repeated until the desired sequence information has been obtained.

The following represents a special embodiment of the method according to the invention, in which, in step (a),

(a1) a surface is provided on which at least primer molecules of a first primer and of a second primer, and, where appropriate, a nucleic acid mixture comprising the nucleic acid molecules with which both primers can hybridize, have been irreversibly immobilized, with the two primers forming a primer pair;

(a2) nucleic acid molecules in the nucleic acid mixture are hybridized with one or both primers of the same primer pair;

(a3) the irreversibly immobilized primer molecules are extended in a complementary manner to the counterstrand, with the formation of secondary nucleic acids;

(a4) the surface is provided in a form which is freed from nucleic acid molecules which are not bound to the surface by irreversible immobilization;

(a5) the secondary nucleic acids are amplified with the formation of tertiary nucleic acids.

Tertiary nucleic acids according to step (a) can be provided by proceeding from a surface on which at least a first primer and a second primer, and, if appropriate, a nucleic acid mixture comprising the nucleic acid modules with which both primers are able to hybridize, have been irreversibly immobilized. The two primers form a primer pair and can consequently bind to the strand and the counterstrand, respectively, of the nucleic acid molecules. When the nucleic acid molecules in the nucleic acid mixture are already bound to the surface, the hybridization in step (a2) can be brought about simply by heating and cooling. Otherwise, the nucleic acid molecules in the nucleic acid mixture have to be brought into contact, in step (a2), with the surface. In this connection, the reader is also referred to WO 00/18957.

The following represents a special embodiment of the method according to the invention, in which, in step (a1),

a surface is provided on which at least primer molecules forming a primer pair have been irreversibly immobilized.

In the case of this embodiment, the individual operational steps which have to be carried out can also be expressed as follows:

primer molecules, which form at least one primer pair, are irreversibly immobilized on a surface;

nucleic acid molecules are hybridized with one or both primers of the same primer pair by bringing the nucleic acid mixture into contact with the surface;

the irreversibly immobilized primer molecules are extended in a complementary manner to the counterstrand, with the formation of secondary nucleic acids;

the nucleic acid molecules which are not bound to the surface by irreversible immobilization are removed from the surface;

the secondary nucleic acids are amplified, with the formation of tertiary nucleic acids;

counterstrands of the tertiary nucleic acids, i.e. TNCs, are provided;

the TNCs are extended by one nucleotide, with

the nucleotide at the 2′-OH position or at the 3′-OH position carrying a protecting group which prevents further extension,

the nucleotide carries a molecular group which enables the nucleotide to be identified;

the incorporated nucleotide is identified;

the protecting group is removed and the molecular group of the incorporated nucleotide, used for identification, is removed or altered, and

the 7th step and the subsequent steps are repeated until the desired sequence information has been obtained.

The nucleic acid mixture in step (a2) can, for example, be a library, that is nucleic acid molecules which possess an identical sequence over long stretches but which differ markedly in a constituent region within the identical regions. The libraries frequently consist of plasmids, which may have been linearized and into which various nucleic acid fragments, which are subsequently sequenced, have been cloned. In addition, the nucleic acid mixture can comprise restriction fragments to the cut ends of which linker molecules having the same sequence have been ligated. In this connection, the linkers which are bonded to the 5′ ends of the fragments as a rule differ from the linkers which are bonded to the 3′ ends of the fragments. At any rate, the sequence segment of interest in the nucleic acid molecules in the nucleic acid mixture is as a rule surrounded by two flanking sequence segments which are essentially in each case identical in all the nucleic acid molecules, with at least one of the two sequence segments preferably possessing a self-complementary sequence. In single-stranded form, the sequence segment in question possesses a marked tendency to form what is termed a hairpin structure.

The primers or the primer molecules in step (a1 to a3) are single-stranded nucleic acid molecules which are from about 12 to about 60 nucleotide building blocks, or more, in length and which are suitable, in the widest possible sense, for use within the context of PCR. They are DNA molecules or RNA molecules, or their analogs, which are intended for hybridizing with a nucleic acid which is complementary over at least a constituent region and which, as a hybrid together with the nucleic acid, constitute a substrate for a double strand-specific polymerase. The polymerase is preferably DNA polymerase I, T7 DNA polymerase, the Klenow fragment of DNA polymerase I, polymerases which are used in PCR, or else reverse transcriptase.

The primer pair in step (a2) constitutes a set of two primers which bind to regions of a nucleic acid which flank the target sequence, which is to be amplified, of the nucleic acid and which exhibit a “polarity” with regard to the orientation in which they are bound to the nucleic acid which is such that amplification is possible (the 3′ termini point towards each other). These regions are preferably sequence sections which are identical in the nucleic acid molecules in the nucleic acid mixture. For example, the nucleic acid mixture can be a plasmid library. The primers would then preferably bind in the region of what is termed the multiple cloning site (MCS), specifically in the one case upstream and in the one case downstream of the cloning site. Furthermore, the primers could bind to the sequence segments which correspond to the linkers which, as described above, have been ligated to the two ends of restriction fragments. The method according to the invention is preferably carried out using only one primer pair, for example like the method described in U.S. Pat. No. 5,641,658 (WO 96/04404), which method also uses only one primer pair. According to the invention, the primers of the primer pair or the primer pairs preferably bind to sequence regions which are essentially identical (what are termed conserved regions) in all or almost all nucleic acids in the nucleic acid mixture. Moreover, the primers in a primer pair can also have the same sequence. This can be advantageous when the conserved regions which flank the sequence to be amplified have sequences which are complementary to each other.

One of the primers in a primer pair can have a sequence which makes it possible to form an intra-molecular nucleic acid double helix (what is termed as a hairpin structure), with, however, a region at the 3′ terminus composed of at least 13 nucleotide building blocks remaining unpaired.

The surface in step (a, a1 and a2, a4) is the accessible area of a body made out of plastic, metal, glass, silicon or similarly suitable materials. The surface is preferably flat, and in particular planar in form. The surface can possess a swellable layer, for example composed of polysaccharides, polysugar alcohols or swellable silicates.

Irreversible immobilization means the formation of interactions with the above-described surface, which interactions are stable, on a scale of hours, at 95° C. and the customary ionic strength in connection with the PCR amplifications in step (a5). The interactions are preferably covalent bonds which can also be cleavable. Preference is given to the primer molecules in step (a) being irreversibly immobilized on the surface by way of the 5′ termini. Alternatively, an immobilization can also be immobilized by way of one or more nucleotide building blocks which lie between the termini of the primer molecule in question, with, however, a sequence segment of at least 13 nucleotide building blocks, calculated from the 3′ terminus, having to remain unbound. The immobilization is preferably effected by forming covalent bonds. In this connection, care has, of course, to be taken to ensure that an appropriate coverage density, which enables the primers and nucleic acids involved in the polymerase chain reaction to make contact with each other, is achieved. If two primers are immobilized, the primers should then have an average distance from each other on the surface which is at least of the same order of magnitude as the maximum length of the nucleic acid molecules to be amplified when completely extended, or is less than this length. The procedure to be followed in this connection is essentially that described in U.S. Pat. No. 5,641,658 or WO 96/04404.

Methods for binding oligonucleotides, which have been suitably derivatized chemically, to glass surfaces are known in the prior art. Terminal primary amino groups (amino link), which are bonded to the 5′ end of the oligonucleotide by way of a multiatom spacer, which can readily be incorporated during the course of the oligonucleotide sythesis, and which are able to react well with isothiocyanate-modified surfaces, are, for example, particularly suitable for this purpose. For example, Guo et al. (Nucleic Acids Res. 22 (1994), 5456-5465) describe a method for activating glass surfaces with aminosilane and phenylene diisothiocyanate and subsequently binding 5′-amino-modified oligonucleotides to these surfaces. The carbodiimide-mediated binding of 5′-phosphorylated oligonucleotides to activated polystyrene supports (Rasmussen et al., Anal. Biochem 198 (1991), 138-142) is particularly suitable. Another known method exploits the high affinity of gold for thiol groups for the purpose of binding thiol-modified oligonucleotides to gold surfaces (Hegner et al, FEBS Lett 336 (1993), 452-456).

The term secondary nucleic acid in step (a3) describes those nucleic acid molecules which are formed as the result of complementary extension of primer molecules, the extension taking place complementary to the nucleic acid molecules of step (a2), which nucleic acid molecules were hybridized with the primers.

The surface is provided in a form which is freed from nucleic acid molecules which are not bound to the surface by irreversible immobilization [step (a4)]. Provided the nucleic acid molecules from step (a1) have already been immobilized irreversibly on the surface in step (a1), no nucleic acid molecules are as a rule brought into contact with the surface in step (a2). Consequently, they do not have to be removed in the following steps, either. If nucleic acid molecules are brought into contact with the surface, for the purpose of hybridization with the primers, in step (a2), for example because the nucleic acid molecules have not already been immobilized irreversibly on the surface in step (a1), these nucleic acid molecules can then be removed, by denaturation and washing, in step (a4). It is possible, though not preferred, only to remove the abovementioned nucleic acid molecules after going through one or more amplification cycles of step (a5).

The term tertiary nucleic acids describes secondary nucleic acids and those nucleic acid molecules which are formed from the secondary nucleic acids in step (a5) by the method of polymerase chain reaction. In this connection, it is important that the surface and the liquid reaction space surrounding the surface are free from nucleic acids which are to be amplified and which are not irreversibly immobilized on the surface. As a rule, the amplification results in the formation of regular islands, that is discrete regions on the surface which carry tertiary nucleic acids of the same type, that is identical nucleic acid molecules or nucleic acid molecules which are complementary to these identical nucleic acid molecules.

Step (b) provides counterstrands of the tertiary nucleic acids (TNCs). This can take place, for example, as the result of one of three measures, which are listed below:

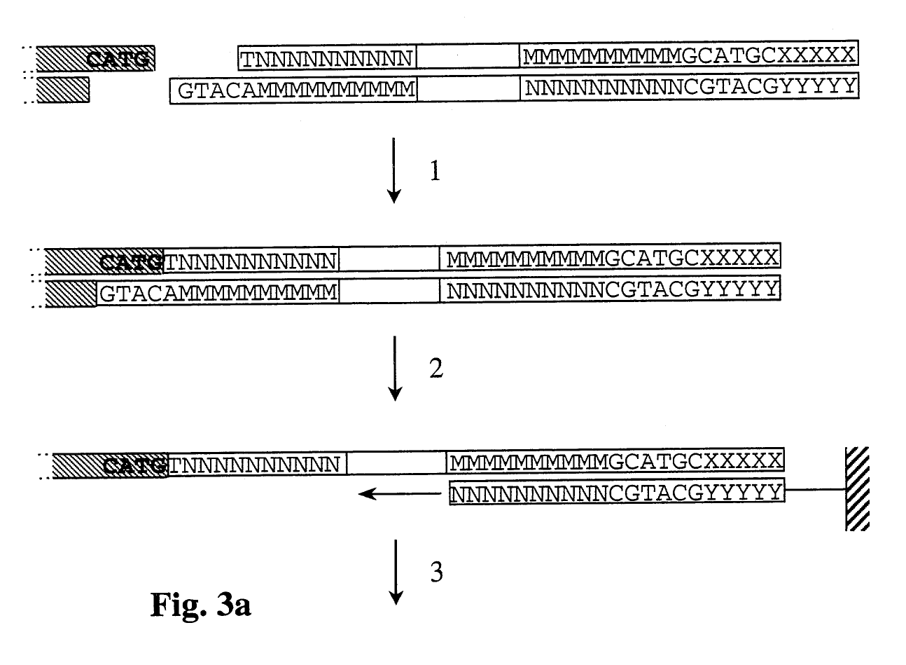

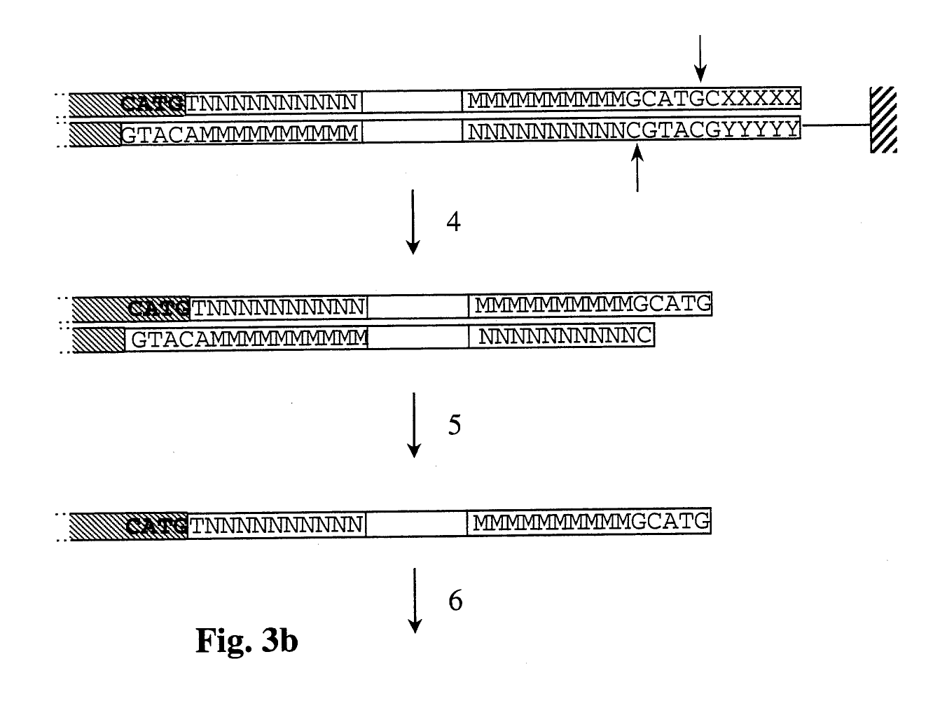

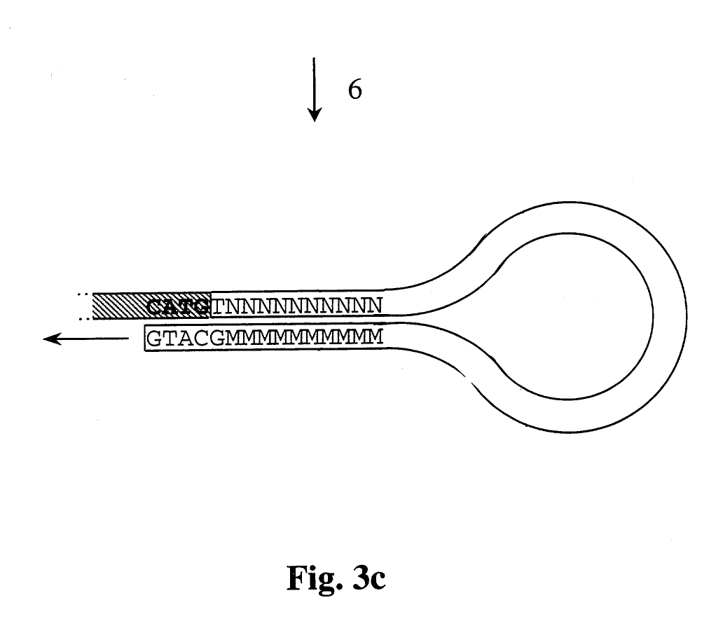

firstly, it is possible to use primer molecules, in step (a1), or, where appropriate, nucleic acid molecules (of the nucleic acid mixture) having flanking sequence segments, in step (a1 or a2), which possess self-complementary regions and are consequently able to carry out intramolecular base-pairing, which is expressed in what is termed a hairpin structure (see also FIG. 3: Ligation of “masked hairpins” in the form of double-stranded linker molecules). In this connection, preference is given to only one primer of a primer pair or only one flanking sequence segment out of two being able to form a hairpin structure in order to ensure that nucleotides are only incorporated at one of two complementary nucleic acid molecules such that the possibility of the sequence signals of the two nucleic acid molecules interfering is excluded.

The tertiary nucleic acids which are formed in step (a5) then exhibit, in the single-stranded state which is brought about by removing one of the two strands under denaturing conditions, a back-folding in the form of a hairpin in the vicinity of their 3′ terminus. Preferably, the double-stranded portion of the hairpin extends up to and including the last base of the 3′ end, such that said hairpin can be used directly as a substrate for a polymerase used for sequencing. This has to be ensured by appropriate selection of the sequence of the primer molecules or of the sequence segments flanking the nucleic acid molecules.

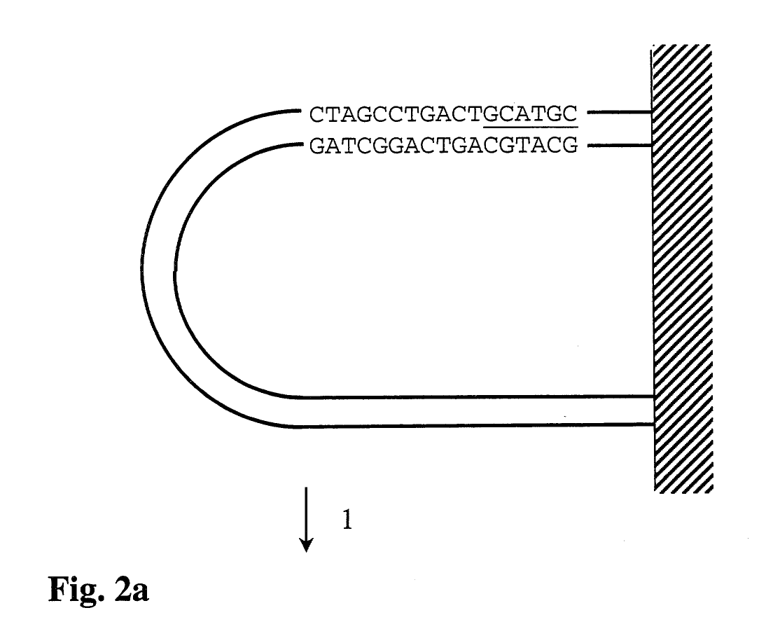

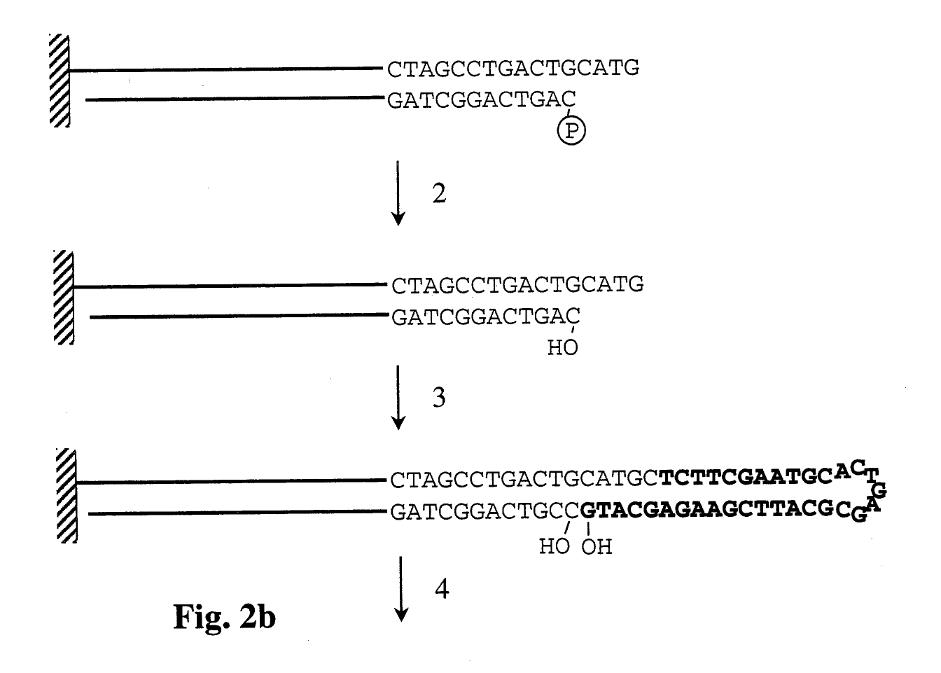

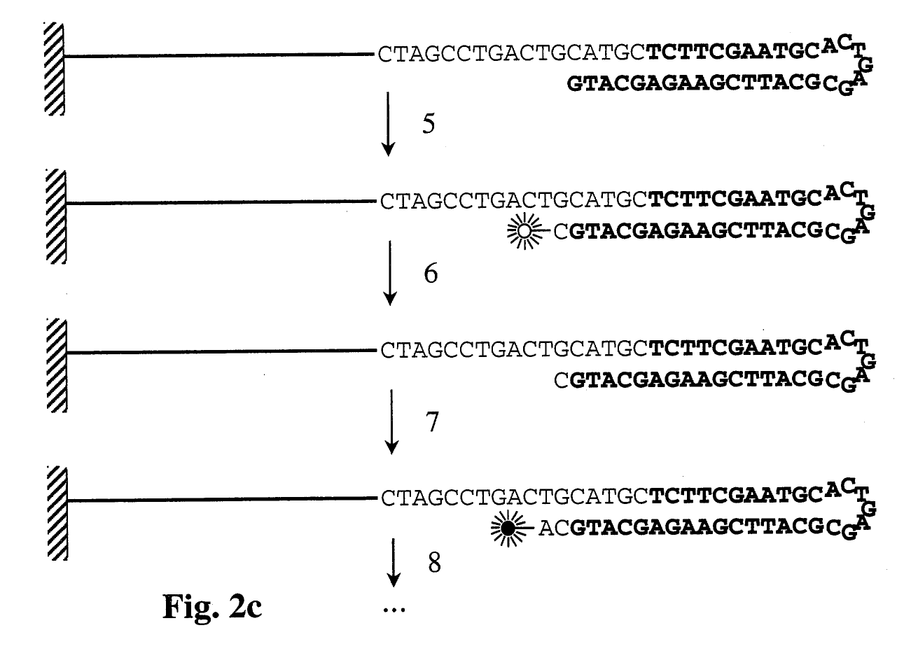

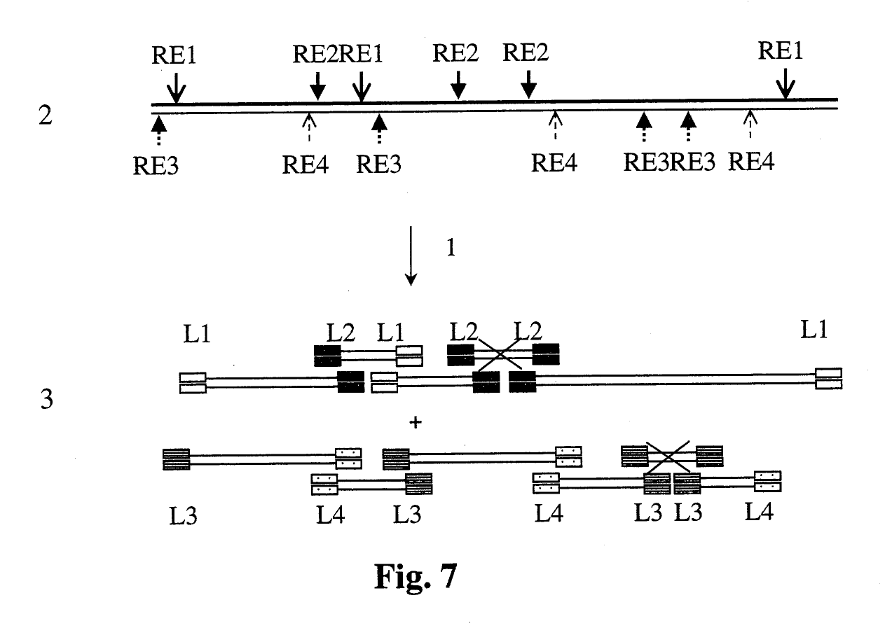

Secondly, TNCs can be provided in the form of hairpins by ligating oligonucleotides which are capable of hairpin formation and, where appropriate (but not necessarily), are already used for ligation in the form of hairpins (see also FIG. 2). This can take place such that the tertiary nucleic acids are cut in the double-stranded (that is undenatured) state and in this way separated at one end from the surface. This preferably takes place by incubating with a restriction endonuclease which possesses a recognition site in precisely one of the sequences derived from one of the two primers (primer sequences) or in a sequence adjoining these primer sequences. After the restriction cleavage has taken place, a free end of the tertiary nucleic acids then protrudes into the solution space, which free end possesses an overhanging end of a sequence which can be predicted depending on the restriction endonuclease employed and to which the oligonucleotide can be hybridized and ligated. An oligonucleotide which has already formed a hairpin structure, and is accordingly therefore present in partially double-stranded form, and possesses an overhang which is complementary to the free end of the tertiary nucleic acids, would be particularly suitable for this purpose. In order to ensure that a ligation takes place exclusively to the irreversibly immobilized strand of the double strand of the tertiary nucleic acids, the 5′ end of the oligonucleotide can carry a phosphate group whereas the 3′ end of the irreversibly immobilized strand and the 5′ end of the counterstrand which is hybridized with this latter strand possess an OH group (see FIG. 2, steps 1 and 2). After ligation has taken place, the strand of the tertiary nucleic acids which is not irreversibly immobilized is removed under denaturing conditions. Alternatively, as proposed in U.S. Pat. No. 5,798,210 (see, in particular, FIG. 7 in this latter publication), an oligonucleotide which has been back folded to form a hairpin could also be ligated to the immobilized strand, which is present in single-stranded form, of the tertiary nucleic acids. A problem in connection with this second measure is that it is no longer possible, as in the case of the first measure, to use amplification steps to compensate for the efficiency of the ligation step prior to sequencing being inadequate, as is frequently observed. This can result in the signal strength in association with the subsequent sequencing being too low.

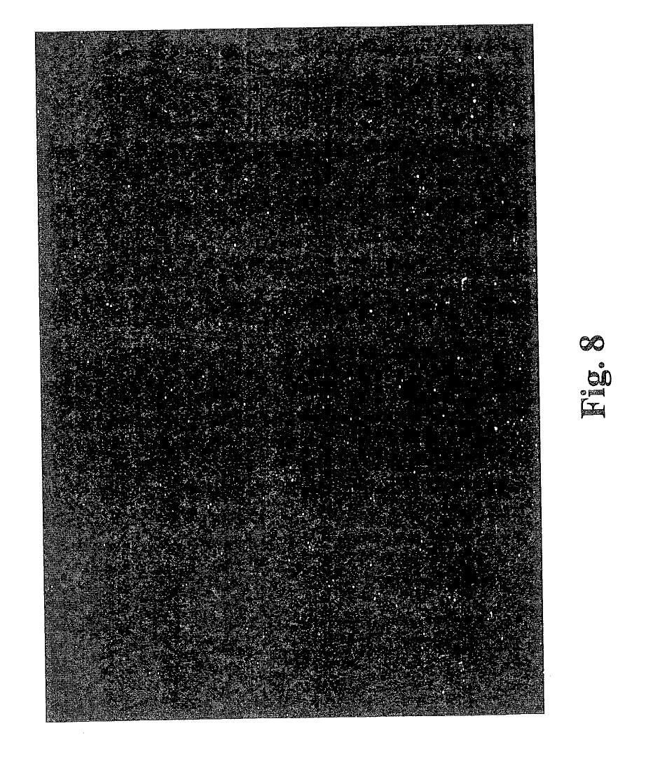

Thirdly, it is also possible to hybridize oligonucleotides which are not able to form a hairpin structure with the tertiary nucleic acids, with the formation of TNCs (cf. U.S. Pat. No. 5,798,210, FIG. 8). This alternative would in any case only come into consideration when, in step (e), in which the protecting group is removed, conditions are selected which do not lead to denaturation, that is which do not lead to melting, of the double strand consisting of oligonucleotides, which have possibly been extended, and tertiary nucleic acids. If step (e) is carried out under denaturing conditions (e.g. as a result of employing relatively strong bases), the other measures are then preferably used.

Within the context of the measures described, the lengths of the oligonucleotides are only of subsidiary importance. As a rule, the oligonucleotides will have a length of less than 100 or less than 50 nucleotide building blocks such that one can also refer to them, in a general manner, as being nucleic acids (in this present case: polymeric nucleotides which comprise more than three nucleotide building blocks). As a result of nonspecific interactions, single-stranded oligonucleotides having a length of more than 45 nucleotide building blocks can only be handled with difficulty when they do not possess any sequence which enables hairpins to be formed. The ability to form hairpins reduces nonspecific interactions by competition. Consequently, the lengths of the oligonucleotides are of hardly any importance when double-stranded polynucleotides are used (see also FIG. 3).

A consequence of the measures described is that the tertiary nucleic acids possess a constituent double-stranded region which enables a DNA polymerase or reverse transcriptase to carry out strand extension on the counterstrands of the tertiary nucleic acids (TNCs).

The nucleotide, which is incorporated in a complementary manner to the counterstrand in step (c) is a termination nucleotide which can be deprotected. Suitable termination nucleotides are disclosed, for example, in U.S. Pat. No. 5,798,210. Canard and Sarfati (Gene 148 (1994) 1-6) describe 3′-esterified nucleotides which contain a fluorophore which can be eliminated together with the protecting group. These nucleotide building blocks can be incorporated by various polymerases, although with low efficiency, into a growing strand, and then act as termination nucleotides; that is they do not permit any further strand extension. The described esters can be cleaved off under alkaline conditions or enzymically, resulting in the formation of free 3′-OH groups which permit further nucleotide incorporation. However, the ester cleavage takes place very slowly (within the space of 2 hours), which means that the described compounds are unsuitable for sequencing relatively long DNA segments (e.g. more than 20 bases). As long as the protecting group is bonded in the 3′-OH or, where appropriate, 2′-OH position (see below), the quaternary nucleic acid which has been extended by this nucleotide no longer constitutes a substrate for a nucleic acid polymerase. It is only the removal of the protecting group in step (e) which makes further extension of the quaternary nucleic acid possible. In addition, the protecting group as a rule carries a molecular group which makes it possible to identify the incorporated nucleotide, and consequently to sequence the growing nucleic acid strand, and which leaves the nucleotide when the protecting group is eliminated. However, the identifying molecular group can also be bonded at another site in the nucleotide, for example at the base. In this case, it is necessary, after step (d), to quench the signal of the identifying molecular group in step (e). As a rule, this can be done in two ways. For example, in the case of a fluorophore, the molecular group can be altered by being bleached out. In addition, the identifying molecular group can also be removed, for example by the photochemical cleavage of a photolabile bond.

If the identifying molecular group is not bonded to the protecting group, and if the identifying molecular group is eliminated for quenching the signal, the bonding of the protecting group to the nucleotide, and the bonding of the identifying molecular group to the nucleotide, are preferably to be selected such that both groups can be eliminated in one reaction step.

Preference is given to each of the four nucleotide building blocks (G, A, T, C) coming into consideration for the incorporation possessing a different identifying molecular group. In this case, the four types of nucleotide can be offered simultaneously in step (c). If different nucleotides, or even all the nucleotides, carry the same identifying molecular group, step (c) then has as a rule to be split into four constituent steps, in which the nucleotides of one type (G, A, T, C) are offered separately.

The molecular group is, for example, a fluorophore or a chromophore. The absorption maximum of the latter could be in the visible frequency range or in the infrared frequency range. The detection which takes place in step (d) is effected in both a site-resolved and time-resolved manner such that the islands of quaternary nucleic acids which are located on the surface can be sequenced in parallel.

A protecting group of the nucleotide in step (c) is to be understood as being a chemical substituent which prevents further strand extension after the nucleotide has been incorporated at its 3′ position. In this connection, the protecting group can occupy the 3′ position which is to be protected, that is be linked to the C-3 of the ribose or screen the 3′ position which is to be protected and in this way sterically prevent strand extension. In the latter case, the protecting group would be linked to the nucleotide in an adjacent position, in particular at the C-2 of the ribose.

In another embodiment of the process according to the invention, primers or nucleic acid molecules possessing flanking sequence segments which exhibit self-complementary regions are used in step (a1).

In another embodiment of the process according to the invention, the tertiary nucleic acids are cut by a restriction endonuclease, in step (b), before oligonucleotides, which are capable of forming a hairpin structure, are ligated to the ends which are generated in this manner. The reader is referred to the comments on step (b), measure 2, on page 9, in particular to the explanation of the term oligonucleotide.

In a further embodiment of the process according to the invention, the oligonucleotides which are capable of forming a hairpin structure are single-stranded. In this present case, single-stranded means not double-stranded throughout. The oligonucleotides are consequently not present as heterodimers. This is the case, for example, in FIG. 2.

In another embodiment of the process according to the invention, the oligonucleotides which are capable of forming a hairpin structure are double-stranded. The oligonucleotides are consequently present as heterodimers. This is the case, for example, in FIG. 3.

In another embodiment of the process according to the invention, single-stranded oligonucleotides which are capable of forming a hairpin structure are hybridized to tertiary nucleic acids, in step (b), before the tertiary nucleic acids and aforementioned single-stranded oligonucleotides are ligated. This is the case, for example, in FIG. 2. In this connection, however, account has to be taken of the fact that the hybrid formation is frequently unstable (e.g. when overhangs consisting of 4 nucleotide building blocks are hybridized), which means that hybrid formation and ligation directly follow one another. In a further embodiment of the process according to the invention, single-stranded oligonucleotides which are capable of forming a hairpin structure are linked to tertiary nucleic acids by ligation in step (b). In this connection, it is also possible to ligate blunt ends. This ligation does not require any prior hybrid formation.

In another embodiment of the process according to the invention, the primer molecules are irreversibly immobilized, in step (a, al), by forming a covalent bond with a surface.

In another embodiment of the process according to the invention, the base carries, in step (c), the molecular group which enables the nucleotide to be identified.

In another embodiment of the process according to the invention, the nucleotide carries the protecting group at the 3′-OH position in step (c).

In a further embodiment of the process according to the invention, the protecting group possesses a cleavable ester, ether, anhydride or peroxide group.

In another embodiment of the process according to the invention, the protecting group is linked to the nucleotide by way of an oxygen-metal bond.

In a further embodiment of the process according to the invention, the protecting group is removed, in step (e), using a complex-forming ion, preferably using cyanide, thiocyanate, fluoride or ethylenediamine tetraacetate.

In another embodiment of the process according to the invention, the protecting group possesses a fluorophore in step (c) and the nucleotide is identified fluorometrically in step (d).

In another embodiment of the process according to the invention, the protecting group is eliminated photochemically in step (e).

The invention is described in more detail by means of the drawing, with the pages of the drawing being numbered consecutively (1/10 to 10/12).

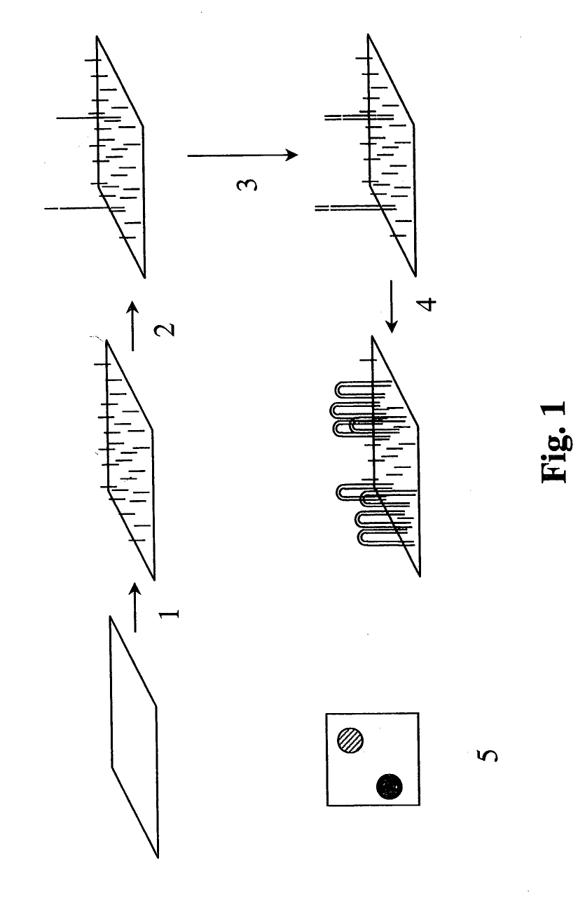

FIG. 1 shows the amplification of individual nucleic acid molecules, using surface-bound primers, to form islands which are in each case composed of identical amplified nucleic acid molecules, with this figure comprising one drawing page (1/12);

FIG. 2 shows the sequencing of surface-bound amplification products, with this figure comprising FIG. 2a, FIG. 2b and FIG. 2c, on drawing pages 2/12 to 4/12;

FIG. 3 shows the preparation of a TNC by forming a hairpin structure in sequence segments which are derived from linkers, with this figure comprising FIG. 3a, FIG. 3b and FIG. 3c on drawing pages 5/12 to 7/12;

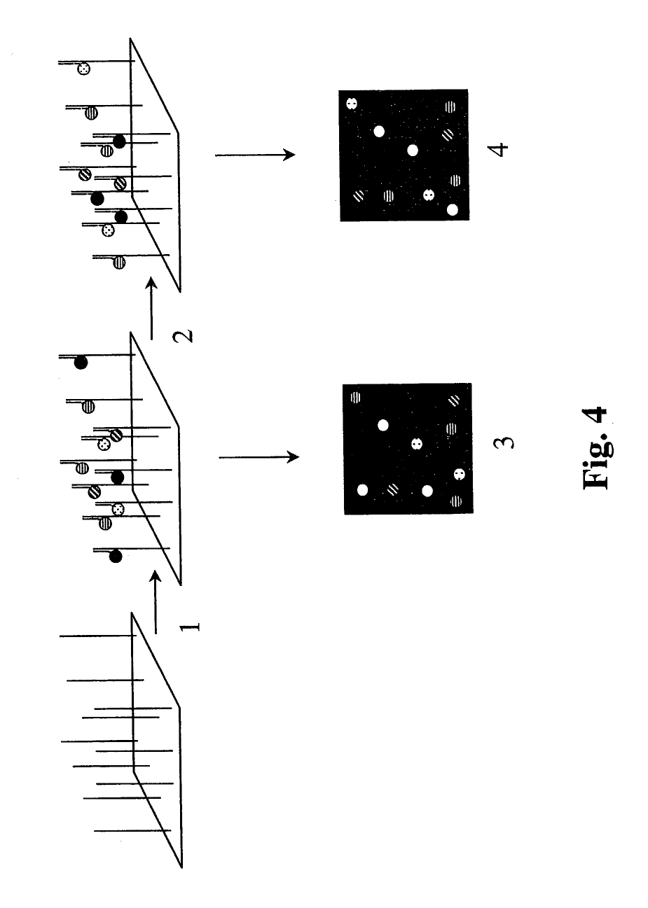

FIG. 4 shows sequencing in parallel on a surface, with this figure comprising one drawing page (8/12);

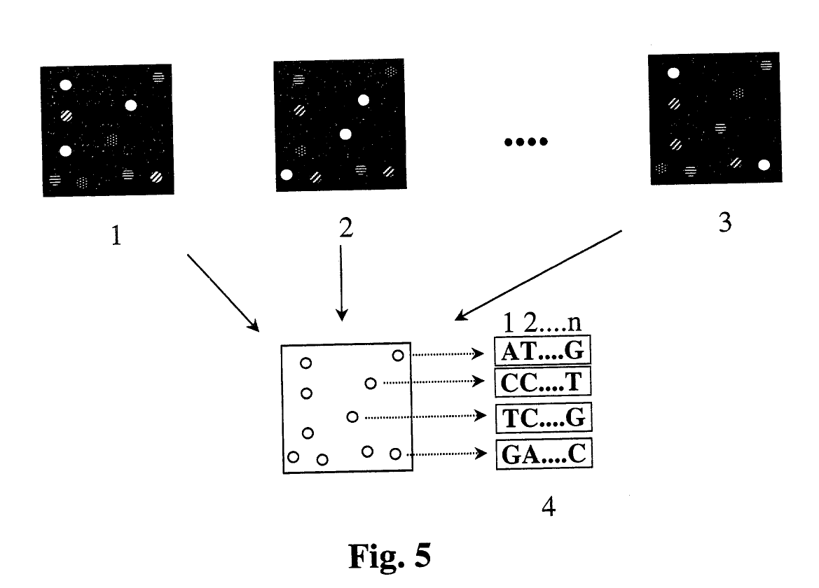

FIG. 5 shows the assembling of the detection and identification results to give contiguous sequences, with this figure comprising one drawing page (9/12);

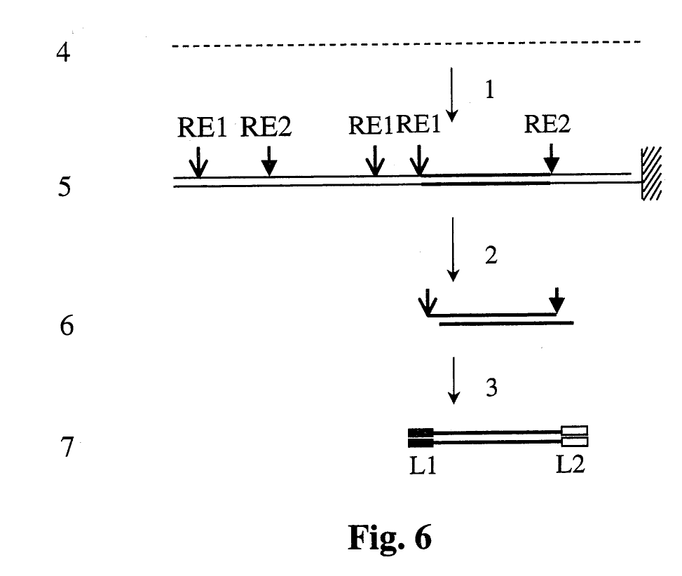

FIG. 6 shows the preparation of primary nucleic acids for use in the expression analysis, with this figure comprising one drawing page (10/12);

FIG. 7 shows the preparation of primary nucleic acids for sequencing genomic clones, this figure comprising one drawing page (11/12);

FIG. 8 shows the result of amplifying individual nucleic acid molecules as shown in FIG. 1, with this figure comprising one drawing page (12/12).

FIG. 1 shows the amplification of individual nucleic acid molecules, using surface-bound primers, to form islands which are in each case composed of identical amplified nucleic acid molecules, with, individually,

1 showing the irreversible immobilization of primer pair-forming oligonucleotides,

2 showing the hybridization of the primary nucleic acids to the surface-bound primers,

3 showing the formation of secondary nucleic acids by strand extension of the primers,

4 showing the removal of the primary nucleic acid molecules which are not irreversibly bound and amplification of the secondary nucleic acids,

5 showing islands which in each case contain identical tertiary nucleic acid molecules.

FIG. 2 illustrates the sequencing of surface-bound amplification products, with

1 showing the use of restriction digestion to release the amplification products (underlined: the recognition site for the restriction endonuclease SphI) (SEQ ID NO: 4);

2 showing the dephosphorylation;

3 showing the ligation of a hairpin oligonucleotide (in bold)

4 showing the removal of the nucleic acid strand which is not irreversibly immobilized (SEQ ID NO: 6);

5 showing the incorporation and identification of a first protected nucleotide;

6 showing the removal of the protecting group and labeling group while at the same time restoring a free 3′-OH group;

7 showing the incorporation and identification of a second protected nucleotide;

8 showing the repetition of steps 5 and 6.

FIG. 3 shows the preparation of a TNC by forming a hairpin structure in sequence segments which are derived from linkers. The nucleic acid which is to be sequenced (restriction fragment possessing two different ends, one of which is generated by the restriction endonuclease NlaIII) is hatched. CATG, overhang generated by the restriction endonuclease NlaIII; GCATGC, recognition site for the restriction endonuclease SphI (contains the recognition site for NlaIII, CATG); NNNNNNNNNN and MMMMMMMMMM, inverted repeats (sequences which are complementary to each other and which permit the intramolecular back-folding of a single strand); XXXXX and YYYYY, spacer region in relation to the surface. Individually,

1 shows the ligation of a linker, containing an inverted repeat and an SphI cleavage site, to a fragment to be sequenced;

2 shows denaturation and hybridization to a primer which is immobilized on a surface;

3 shows amplification using two primers which are immobilized on the surface (counter primer not shown);

4 shows the “one-ended release” of the amplification products from the surface using restriction endonuclease SphI (arrows);

5 shows the denaturation and removal of the strand which is not immobilized on the surface;

6 shows renaturation, with the formation of a hairpin, and the beginning of the sequencing by incorporating deprotectable termination nucleotides.

FIG. 3 shows a preferred procedure for preparing tertiary nucleic acid counterstrands, i.e. TNCs, which serve as sequencing primers, by initially using a ligation to provide the nucleic acid molecules, which have been equipped with overhanging ends by being treated with a first restriction endonuclease (having the recognition sequence CATG in FIG. 3, for example), with flanking sequence segments in the form of double-stranded linker molecules which firstly comprise self-complementary regions and secondly possess, distally adjacent to these, a recognition sequence or cleavage site for a second restriction endonuclease. Preferably, this cleavage site is, as shown in FIG. 3, a cleavage site whose inner bases on the same strand are identical to the bases in said overhang (the base sequence CATG in FIG. 3), with, however, at least one of the outer bases differing from the corresponding base flanking said overhang sequence before or after ligation. For example, FIG. 3 shows that the overhang “CATG” used for the ligation is flanked by the base “T” at its 3′ end after the ligation. If now, after the nucleic acid molecules have been amplified in step (a5) using a primer pair, one primer of which can hybridize with a strand of said linker molecules, cutting is performed using a second restriction endonuclease which, for example, possesses the recognition sequence “GCAGTC”, and if this recognition sequence was provided in said flanking sequence segments (that is as a constituent sequence of the attached linkers), a cut then takes place within the provided sequence segments. After the strand which is then no longer irreversibly bound to the surface has been removed, the 3′ terminus of the strand which remains immobilized can fold back intramolecularly to form a hairpin. In this connection, preference is given, as shown in FIG. 3, to the recognition sequence of said first and second restriction endonucleases directly adjoining the self-complementary regions which have been introduced, such that these regions are extended, by means of said ligation, by the bases which are common to the two said recognition sites. In this connection, the extended self-complementary regions exhibit a mispairing where, after ligation, the base (or bases) flanking the overhang sequence differ(s) from the recognition sequence for the second restriction endonuclease (a G/T mispairing in FIG. 3). At the same time, the procedure which is described here, in which the recognition site for the first restriction endonuclease is a component of the longer recognition site for the second restriction endonuclease, ensures that, in connection with incubating with the second restriction endonuclease, the tertiary nucleic acid molecules cannot possess any internal recognition sites for the second restriction endonuclease but, instead, are only cut precisely once in the region of the flanking sequences.

FIG. 4 describes sequencing in parallel on a surface. For simplicity, “islands” of identical nucleic acid molecules are symbolized in this figure by means of a single strand. Individually,

1 shows the attachment of a sequencing primer, incorporation of the first termination nucleotide and parallel detection and identification of the first nucleotide building block in each case,

2 shows the removal of the protecting group and labeling group of the first nucleotide, incorporation of the second termination nucleotide and parallel detection and identification of the second nucleotide building block in each case;

3 shows the detection and identification result for the first base;

4 shows the detection and identification result for the second base.

FIG. 5 describes the assembling of the detection and identification results to give contiguous sequences, where

1 shows the detection and identification results for the first base,

2 shows the detection and identification results for the second base,

3 shows the detection and identification results for the nth base,

4 shows the assembled sequences of the nucleic acid molecules in individual islands.

FIG. 6 shows the preparation of primary nucleic acids for use in the expression analysis, with, individually,

1 showing cDNA synthesis using a biotinylated primer, binding of the double-stranded cDNA to a streptavidin-coated surface;

2 showing the restriction cleavage with the first enzyme (REl), washing-away of the released fragments and the second restriction cleavage with the second enzyme (RE2);

3 showing the ligation of two different linkers;

4 showing mRNA;

5 showing double-stranded cDNA which is immobilized to a solid phase;

6 showing a cDNA fragment which is flanked by two different “overhanging” ends,

7 showing a cDNA fragment which is flanked by two different linkers (L1 and L2).

FIG. 7 shows the preparation of primary nucleic acids for sequencing genomic clones, with, individually,

1 showing a parallel restriction cleavage of a genomic clone with in each case two different restriction endonucleases (RE1-2 and RE3-4, respectively), and the ligation of various linkers (L1-4);

2 showing a genomic clone;

3 showing two overlapping sets of fragments ligated to the linkers.

Fragments (deleted) which are symmetrically flanked by identical linkers cannot be sequenced.

FIG. 8 shows the result of using surface-bound primers to amplify individual nucleic acid molecules to form islands which in each case consist of identical amplified nucleic acid molecules, as visualized by staining with SYBR Green I.

The examples which follow clarify the invention.

### EXAMPLE 1

Preparing Nucleic Acid Molecules

4 μg of total RNA from rat liver were precipitated with ethanol and dissolved in 15.5 μl of water. 0.5 μl of 10 μM cDNA primer CP28V (5′-ACCTACGTGCAGATTTTTTTTTTTTTTTTTTV-3′, SEQ ID No:1) was added and the mixture was denatured at 65° C. for 5 minutes and placed on ice. 3 μl of 100 mM dithiothreitol (Life Technologies GmbH, Karlsruhe), 6 μl of 5×Superscript buffer (Life Technologies GmbH, Karlsruhe), 1.5 μl of 10 mM dNTPs, 0.6 μl of RNase inhibitor (40 U/μl; Roche Molecular Biochemicals) and 1 μl of Superscript II (200 U/μl, Life Technologies) were added to the mixture, which was incubated at 42° C. for 1 hour for synthesizing the first cDNA strand. For synthesizing the second strand, 48 μl of second-strand buffer (cf. Ausubel et al., Current Protocols in Molecular Biology (1999), John Wiley & Sons), 3.6 μl 10 mM dNTPs, 148.8 μl of H₂O, 1.2 μl of RNaseH (1.5 U/μl, Promega) and 6 μl of DNA polymerase I (New England Biolabs GmbH Schwalbach, 10 U/μl) were added and the reactions were incubated at 22° C. for 2 hours. The mixture was extracted with 100 μl of phenol and then with 100 μl of chloroform and precipitated with 0.1 vol. of sodium acetate, pH 5.2, and 2.5 vol. of ethanol. After having been centrifuged at 15,000 g for 20 minutes, and washed with 70% ethanol, the pellet was dissolved in a restriction mixture composed of 15 μl of 10×universal buffer, 1 μl of MboI and 84 μl of H₂O and the reaction was incubated at 37° C. for 1 hour. The mixture was extracted with phenol and then with chloroform and precipitated with ethanol. The pellet was dissolved in a ligation mixture composed of 0.6 μl of 10×ligation buffer (Roche Molecular Biochemicals), 1 μl of 10 mM ATP (Roche Molecular Biochemicals), 1 μl of linker ML2025 (prepared by hybridizing oligonucleotides ML20 (5′-TCACATGCTAAGTCTCGCGA-3′, SEQ ID NO: 5) and LM25 (5′-GATCTCGCGAGACTTAGCATGTGAC-3′, SEQ ID NO: 7), ARK), 6.9 μl of H₂O and 0.5 μl of T4 DNA ligase (Roche Molecular Biochemicals), and the ligation was carried out overnight at 16° C. The ligation reaction was made up to 50 μl with water, after which it was extracted with phenol and then with chloroform; it was then precipitated, after having added 1 μl of glycogen (20 mg/ml, Roche Molecular Biochemicals), with 50 μl of 28% polyethylene glycol 8000 (Promega)/10 mM MgCl₂. The pellet was washed with 70% ethanol and taken up in 100 μl of water.

### EXAMPLE 2

Coating with Oligonucleotides

Lyophilized oligonucleotides carrying amino link groups at their 5′ end, i.e. amino-M13rev (5′-amino-CAGGAAACAGCGATGAC-3′, SEQ ID NO: 8) and amino-T7 (5′-amino-TAATACGACTCACTATAGG-3′, SEQ ID NO: 10) (ARK Scientific GmbH, Darmstadt), were taken up in 100 mM sodium carbonate buffer, pH 9, to give a final concentration of 1 mM. Glass microscope slides (“Slides”; neoLab Migge Laborbedarf-Vertriebs GmbH, Heidelberg) were cleaned for 1 hour in chromic acid and, after that, washed 4×with distilled water. After having been dried in air, the slides were treated for 5 minutes in a 1% strength solution of 3-aminopropyltrimethoxysilane (“Fluka”: Sigma Aldrich Chemie GmbH, Seelze) in 95% acetone/5% water. After that, they were washed ten times, for in each case 5 minutes, in acetone and heated at 110° C. for 1 hour. The slides were then placed for 2 hours in 0.2% 1,4-phenylene diisothiocyanate (“Fluka”: Sigma Aldrich Chemie GmbH, Seelze) in a solution of 10% pyridine in dry dimethylformamide (Merck KGaA, Darmstadt). After 5 washing steps in methanol and 3 washing steps in acetone, the slides were dried in air and directly processed for coating. Small, self-adhering “Frame Seal” frames for 65 μl reaction chambers (MJ Research Inc., Watertown, Minn., USA) were applied, after which 65 μl of oligonucleotide solution were pipetted into the reaction chambers which had been formed in this way; the chambers were then sealed, while excluding air bubbles, by affixing a polyester covering sheet (MJ Research Inc.). The precise position of the reaction chamber was marked on the underside of the slides using a water-resistant felt-tip pen. The oligonucleotides were bound, by way of the amino link, to the surfaces of the activated slides at 37° C. over a period of 4 hours. The adhering frames were then removed and the slides were rinsed with deionized water. In order to inactivate any remaining reactive groups, the slides were treated for 15 minutes in blocking solution (50 mM ethanolamine (“Fluka”: Sigma Aldrich Chemie GmbH, Seelze), 0.1 M Tris, pH 9 (“Fluka”; Sigma Aldrich Chemie GmbH, Seelze), 0.1% SDS (“Fluka”: Sigma Aldrich Chemie GmbH, Seelze) which had been brought to a temperature of 50° C. In order to remove noncovalently bound oligonucleotides, the slides were boiled for 5 minutes in 800 ml of 0.1×SSC/0.1% SDS (cf. Ausubel et al., Current Protocols in Molecular Biology (1999), John Wiley & Sons). The slides were washed with deionized water and air-dried.

### EXAMPLE 3

Coating with Oligonucleotides

Lyophilized oligonucleotides carrying amino link groups at their 5′ end, i.e. amino-Ml3rev (5′-amino-CAGGAAACAGCGATGAC-3′, nucleotide sequence as depicted in SEQ ID NO: 8) and amino-T7 (5′-amino-TAATACGACTCACTATAGG-3′, nucleotide sequence as depicted in SEQ ID NO: 10) (ARK Scientific GmbH, Darmstadt) were taken up in deionized water to give a final concentration of 100 pmol/μl. In each case, 1.4 μl of these primer solutions were mixed with 32.2 μl of water and 35 μl of 2×binding buffer (300 mM sodium phosphate, pH 8.5). Small self-adhering “Frame Seal” frames for 65 μl reaction chambers (MJ Research Inc., Watertown, Minn., USA) were applied to “3D-link activated slides” (glass slide activated for binding amino-modified nucleic acids; (Surmodics, Eden, Prairie, Minn., USA)). 65 μl of oligonucleotide solution were pipetted into the reaction chambers which have been formed in this way and the chambers were sealed, while excluding air bubbles, by affixing a polyester covering sheet (MJ Research Inc., Watertown, Minn., USA). The precise position of the reaction chamber was marked on the underside of the slides using a water-resistant felt-tip pen. The oligonucleotides were bound, by way of amino link, to the surfaces of the activated slides at room temperature overnight. The adhering frames were then removed and the slides were rinsed with deionized water. In order to inactivate any remaining reactive groups, slides were treated for 15 minutes in blocker solution (50 mM ethanolamine (“Fluka”: Sigma Aldrich Chemie GmbH, Seelze), 0.1 M Tris, pH 9 (“Fluka”: Sigma Aldrich Chemie GmbH, Seelze), 0.1% SDS (“Fluka”: Sigma Aldrich Chemie GmbH, Seelze), which had been brought to a temperature of 50° C. In order to remove noncovalently bound oligonucleotides, the slides were boiled for 5 minutes in 800 ml of 0.1×SSC/0.1% SDS (cf. Ausubel et al., Current Protocols in Molecular Biology (1999), John Wiley & Sons). The slides were washed with deionized water and air-dried.

### EXAMPLE 4

Plasmids pRNODCAB (contains bases 982 to 1491 of the rat ornithine decarboxylase transcript, AC number J04791, cloned in the vector pCR II (Invitrogen BV, Groningen, Netherlands) and PRNHPRT (contains bases 238 to 720 of the rat hypoxanthine phosphoribosyl transferase transcript, AC number M63983, cloned in vector pCR II (Invitrogen)) were linearized by in each case 1 μg of plasmid being incubated in a volume of 20 μl 1×restriction buffer H (“Roche Molecular Biochemicals”: Roche Diagnostics GmbH, Mannheim) with in each case 5 U of the restriction enzymes BglII and ScaI (Roche Molecular Biochemicals) at 37° C. for 1.5 hours. Subsequently, the vector insert was amplified by adding 4 μl of 10 mM primer T7 (5′-TAATACGACTCACTATAGG-3′, SEQ ID NO: 10), 4 μl of 10 mM primer M13 (5′-CAGGAAACAGCGATGAC-3′, SEQ ID NO: 8) (ARK), 4 μl of 50 mM MgCl₂ (“Fluka”: Sigma Aldrich Chemie GmbH, Seelze), 5 μl of dimethyl sulfoxide (“Fluka”: Sigma Aldrich Chemie GmbH, Seelze), 1 μl of 10 mM dNTPs (Roche Molecular Biochemicals), and 1 μl of AmpliTaq DNA polymerase (5 u/μl; Perkin-Elmer) to in each case 1 μl of the restriction mixtures in a volume of 100 μl of PCR buffer II (Perkin-Elmer, Foster City, Calif., USA). Subsequently, the reactions were subjected, in a Gene Amp 9700 Thermocycler (Perkin-Elmer), to a temperature program consisting of 20 cycles of denaturation for 20 seconds at 95° C., primer annealing for 20 seconds at 55° C. and primer extension for 2 minutes at 72° C. The amplification products were investigated electrophoretically, on a 1.5% agarose gel, to ensure that they are of the correct size. In order to remove unincorporated primers, the reactions were purified using QiaQuick columns (Qiagen AG, Hilden) in accordance with the manufacturer's instructions, and eluted in 50 μl of deionized water.

### EXAMPLE 5

Amplification

In order to attach the nucleic acids prepared in Example 2 to glass supports, annealing mixtures composed of in each case 1 μl of undiluted amplification product solutions, or of amplification product solutions diluted 1:10, 1:100 and 1:1000 with water in parallel mixtures, in each case 4 μl of 50 mM MgCl₂ solution, in each case 1 μl of bovine serum albumin (20 mg/ml; Roche Molecular Biochemicals), in each case 5 μl of dimethyl sulfoxide, in each case 1 μl of 10 mM dNTPs and in each case 1 μl of AmpliTaq in a total volume of in each case 100 μl of lx PCR buffer II, were produced. While referring to the felt-tip pen markings made on the underside of the slides, frame-seal chambers were applied to the slides prepared in Example 1 in the positions used for the oligonucleotide coating. In each case 65 μl of the annealing mixtures were then pipetted into the reaction chambers and the chambers were then sealed as above. The slides were placed on the heating block of a UNO II in-situ Thermocycler (Biometra biomedizinische Analytik GmbH, Göttingen), covered with a cushion made out of paper towels and pressed onto the heating block using the vertically adjustable heating lid. The following temperature program was used for the annealing and the subsequent primer extension: denaturing for 30 seconds at 94° C., annealing for 10 minutes at 55° C., primer extension for 1 minute at 72° C. After the reaction had been completed, the reaction chambers were removed and the slides were rinsed with deionized water. The slides were then boiled for 1 minute in 800 ml of 0.1×SSC/0.1% SDS, in order to remove the non-covalently bound strands, after which they were rinsed with water and air-dried. In order to perform a compartmentalized amplification of the nucleic acid molecules bound to the support, reaction chambers were once again applied at the previously selected positions and loaded with 65 pi of an amplification mixture having the following composition: 4 μl of 50 mM MgCl₂, 1 μl of bovine serum albumin (20 mg/ml), 5 μl of dimethyl sulfoxide, 1 μl of AmpliTaq (5 U/μl), 1 μl of 10 mM dNTPs, in 100 μl of lx PCR buffer II. After the chambers had been sealed, the following temperature program was used in the in-situ Thermocycler: denaturation for 20 seconds at 93° C., primer annealing for 20 seconds at 55° C., extension for 1 minute at 72° C., for 50 cycles. After the amplification had come to an end, the chambers were removed and the slides were rinsed with water and air-dried. In order to detect the clonal islands which had been formed by the compartmentalized amplification, 40 μl of SYBR green I solution (molecular probes; 1:10,000 in water) were pipetted onto the slides, which were then covered with #2 cover slips (MJ). The detection was performed on a confocal TCS-NT microscope (Leica Microsystems Heidelberg GmbH, Heidelberg) at an excitation wavelength of 488 nm and a detection wavelength of 530 nm. It was possible to detect clonal islands of compartmentalized, amplified nucleic acid molecules, which were distributed over the surface of the slide, in the region of the reaction chambers, in a random array (cf. FIG. 8). On the other hand, no signals originating from the clonal islands were detected in the region of reaction chambers in which, as negative controls, either no oligonucleotides had been bound to the support or the amplification reaction had been performed without any prior hybridization of template molecules. Furthermore, comparison of the slide surfaces in the region of reaction chambers in which different concentrations of template had been used demonstrated a clear dependence of the number of clonal islands formed on the quantity of molecules employed.

### EXAMPLE 6

In order to identify the nucleic acid molecules in the detected clonal islands, the slides were destained for 10 minutes in water after the SYBR green-stained double-stranded DNA had been detected. Reaction chambers were then once again adhered at the same positions as before and a reaction mixture, composed of 12 μl of 10×Universal buffer (Stratagene GmbH, Heidelberg), 1 μl of bovine serum albumin, 3 μl of restriction endonuclease MboI (1 U/μl; Strategene) and 64 μl of water, was pipetted into them. The slides were then incubated at 37° C. for 1.5 h, in order to restrict the nucleic acid molecules using the internal MboI cleavage site, after which the reaction chambers were removed and the slides were washed with water. The strand fragments which were not bound covalently to the glass support were removed by denaturing for two minutes in 800 ml of boiling 0.1×SSC/0.1% SDS. After the slides had been washed once again in water, and air-dried, new reaction chambers were applied. A hybridization solution composed of 8 μl of 10×PCR buffer II, 3.2 μl of 50 mM MgCl₂, 2 μl of 100 pmol/μl oligonucleotide probe Cy5-HPRT (5′-Cy5-TCTACAGTCATAGGAATGGACCTATCACTA-3′, SEQ ID NO: 3; ARK), 2 μl of 100 pmol/μl oligonucleotide probe Cy3-ODC (5′-Cy3-ACATGTTGGTCCCCAGATGCTGGATGAGTA-3′, SEQ ID NO: 2) and 65 μl of water was prepared per hybridization experiment. For each hybridization experiment, 65 μl of the solution were added to the respective reaction chamber and hybridization was carried out at 50° C. for 3 hours. After the end of the hybridization, the reaction chambers were removed and the slides were washed at room temperature for 5 minutes in 30 ml of 0.1×SSC/0.1% SDS. The slides were briefly rinsed with distilled water, air-dried and then used for the detection. The detection was effected as described above, using a confocal laser microscope. The excitation wavelengths employed were 568 nm and 647 nm and signals were detected at 600 nm and 665 nm. It was possible to demonstrate that some of the clonal islands which had previously been detected with SYBR green were detected by the Cy3-ODC probe while some were detected by the Cy5-HPRT probe.

### EXAMPLE 7

Analyzing expression by sequencing nucleic acid molecules in a highly parallel manner The ligation product obtained from Example 1 were diluted 1:1000 with water and 1 μl of this dilution was amplified in a compartmentalized manner for 50 cycles, as described in Example 5. Glass slides coated with the amplification primers amino-CP28V (5′-amino-ACCTACGTGCAGATTTTTTTTTTTTTTTTV-3′, nucleotide sequence as depicted in SEQ ID NO: 1) and amino-ML20 (5′-amino-TCACATGCTAAGTCTCGCGA-3′, nucleotide sequence as depicted in SEQ ID NO: 5), as described, were used for this purpose. In order to release the amplification products unilaterally from the support, the amplification mixture was replaced with a restriction mixture, consisting of 12 μl of 10×Universal buffer (Stratagene), 1 μl of bovine serum albumin and 4 μl of restriction endonuclease MboI in a final volume of 65 μl. After incubating at 37° C. for 2 h, the restriction mixture was replaced with a dephosphorylation mixture consisting of 1 U of arctic crab alkaline phosphatase (Amersham) in 65 μl of the concomitantly supplied reaction buffer. After incubating at 37° C. for 1 hour, and inactivating at 65° C. for 15 minutes, the reaction chambers and the dephosphorylation mixture were removed and the slides were washed thoroughly with distilled water; reaction chambers were applied once again and filled with 65 μl of a ligation mixture comprising 3 U of T4 DNA ligase (Roche Diagnostics) and 500 ng of the 5′-phosphorylated hairpin sequencing primer SLP33 (5′-TCTTCGAATGCACTGAGCGCATTCGAAGAGATC-3′, SEQ ID NO: 9) in 65 μl of the concomitantly supplied ligation buffer. Ligation was carried out at 16° C. for 14 hours, after which the ligation mixture and the reaction chambers were removed. In order to remove the strand fragments which were not bound covalently to the glass support, the slides were treated for 2 minutes in 800 ml of boiling 0.1×SSC/0.1% SDS and then washed with distilled water. In order to prepare suitable, deprotected termination nucleotides, DATP, dCTP, dGTP and dTTP (Roche Molecular Biochemicals) were esterified at their 3′-OH groups with 4-aminobutyric acid. These derivatives were labeled with the fluorescent groups FAM (dATP and dCTP) and ROX (dGTP and dTTP) (Molecular Probes Inc., Eugene, Oreg., USA). In order to determine the first base in parallel, reaction chambers were once again applied to the slides and filled with a primer extension mixture comprising 1 mM FAM-DATP, 1 mM ROX-dGTP and 2 U of sequenase (United States Biochemical Corp., Cleveland, Ohio, USA) in 65 μl of reaction buffer (40 mM Tris-HCl, pH 7.5, 20 mM of MgCl₂ and 25 mM NaCl). After having been incubated at 37° C. for 5 minutes, the slides were washed with reaction buffer and detection was performed on the laser scanning microscope. The excitation wavelengths were 488 nm and 568 nm, while detection was carried out at 530 nm and at 600 nm. After the detection, primer extension mixture was once again added, with this mixture now containing the remaining two labeled nucleotides, i.e. FAM-dCTP and ROX-dTTP. After incorporation had taken place, washing and detection were once again carried out and the protection groups were removed by enzymic cleavage. For this, the slides were treated, at 35° C. for 1 h, with 5 mg of chirazyme L lipase/ml (Roche Diagnostics) in 100 mM potassium phosphate buffer, pH 9. The sequencing was subsequently carried out for a further 15 cycles, as described above.

1. A method for sequencing in parallel at least two different nucleic acids present in a nucleic acid mixture, whereby a surface is produced comprising islands of nucleic acids of in each case the same type by the following steps:
(a1) provision of a surface, on which at least primer molecules of a first primer and of a second primer, and, where appropriate, a nucleic acid mixture comprising the nucleic acid molecules with which both primers can hybridize, have been irreversibly immobilized, with the two primers forming a primer pair;
(a2) hybridisation of the nucleic acid molecules in the nucleic acid mixture with one or with both primers of the same primer pair;
(a3) extension of the irreversibly immobilized primer molecules in a complementary manner to the counterstrand, with the formation of secondary nucleic acids;
(a4) provision of the surface in a form which is freed from nucleic acid molecules which are not bound to the surface by irreversible immobilization;
(a5) amplification of the secondary nucleic acids in the formation of tertiary nucleic acids, whereby said islands of nucleic acids are defined as discrete locations of tertiary nucleic acids of in each case the same type and whereby the tertiary nucleic acids which are immobilzed on this surface are sequenced in parallel by the following steps:
(b) provision of counterstrands of the tertiary nucleic acids, i.e. TNCs,
(c) extension of the TNCs by one nucleotide, with
the nucleotide at the 2′-OH position or at the 3′-OH position carrying a protecting group which prevents further extension,
the nucleotide carrying a molecular group which enables the nucleotide to be identified;
(d) identification of the incorporated nucleotide,
(e) removal of the protecting group and removal or alteration of the molecular group of the incorporated nucleotide, which is used for identification, (f) repetition of step (c), and of the subsequent steps until the desired sequence information has been obtained.

2. Method as claimed in claim 1, characterized in that, in step (a1) a surface is provided on which primer molecules forming at least one primer pair are irreversibly immobilized, and characterized in that, in step (a2) nucleic acid molecules of the mixture of nucleic acid molecules are hybridized with one or both primers of the same primer pair by contacting the mixture of nucleic acid molecules with the surface.

3. The method as claimed in claim 1, characterized in that, in step (a1),
a surface is provided, on which at least primer molecules forming a primer pair have been irreversibly immobilized.

4. The method as claimed in claim 1, characterized in that, in step (a1), use is made of primers or nucleic acid molecules possessing flanking sequence segments which possess self-complementary regions.

5. The method as claimed in claim 1, characterized in that, in step (b), the tertiary nucleic acids are cut with a restriction endonuclease before oligonucleotides, which are capable of forming a hairpin structure, are ligated to the ends which have been generated in this manner.

6. The method as claimed in claim 5, characterized in that the oligonucleotides capable of forming a hairpin structure are single-stranded.

7. The method as claimed in claim 5, characterized in that the oligonucleotides capable of forming a hairpin structure are double-stranded.

8. The method as claimed in claim 1, characterized in that, in step (b), single-stranded oligonucleotides which are capable of forming a hairpin structure are hybridized to tertiary nucleic acids before tertiary nucleic acids and previously mentioned single-stranded oligonucleotides are ligated.

9. The method as claimed in claim 1, characterized in that, in step (b), single-stranded oligonucleotides which are capable of forming a hairpin structure are linked to tertiary nucleic acids by ligation.

10. The method as claimed in claim 1, characterized in that, in step (a1), the primer molecules are irreversibly immobilized on a surface by forming a covalent bond.

11. The method as claimed in claim 1, characterized in that, in step (c), the base carries the molecular group which enables the nucleotide to be identified.

12. The method as claimed in claim 1, characterized in that, in step (c), the protecting group carries the molecular group which enables the nucleotide to be identified.

13. The method as claimed in claim 1, characterized in that, in step (c), the nucleotide carries the protecting group at the 3′-OH position.

14. The method as claimed in claim 1, characterized in that, in step (c), the nucleotide carries the protecting group at the 2′-OH position.

15. The method as claimed in claim 1, characterized in that, in step (c), the protecting group possesses a cleavable ester, ether, anhydride or peroxide group.

16. The method as claimed in claim 1, characterized in that, in step (c), the protecting group is linked to the nucleotide by way of an oxygen-metal bond.

17. The method as claimed in claim 16, characterized in that, in step (e), the protecting group is removed using a complex-forming ion, preferably using cyanide, thiocyanate, fluoride or ethylenediamine tetraacetate.

18. The method as claimed in claim 1, characterized in that, in step (e), the protecting group is eliminated photochemically.

19. The method as claimed in claim 1, characterized in that, in step (c), the protecting group possesses a fluorophore and the nucleotide is identified fluorimetrically in step (d).


SEQUENCE LISTING (10 sequences)

SEQ ID NO: 1 - DNA, 32 bp, Artificial Sequence
  Synthetic oligonucleotide designated CP28V
acctacgtgcagattttttttttttttttttv

SEQ ID NO: 2 - DNA, 30 bp, Artificial Sequence
  oligonucleotide probe designated Cy3-ODC from ARK Scientific, Darmstadt
acatgttggtccccagatgctggatgagta

SEQ ID NO: 3 - DNA, 30 bp, Artificial Sequence
  oligonucleotide probe Cy5-HPRT from ARK Scientific, Darmstadt
tctacagtcataggaatggacctatcacta

SEQ ID NO: 4 - DNA, 17 bp, Artificial Sequence
  hypothetical sequence for illustrating step 1 of the application in Fig. 2
ctagcctgactgcatgc

SEQ ID NO: 5 - DNA, 20 bp, Artificial Sequence
  Oligonucleotide ML20 from ARK Scientific, Darmstadt, starting material for preparing the linker ML2025
tcacatgctaagtctcgcga

SEQ ID NO: 6 - DNA, 51 bp, Artificial Sequence
  hypothetical sequence for illustrating step 4 of the application in Fig. 2
ctagcctgactgcatgctcttcgaatgcactgagcgcattcgaagagcatg

SEQ ID NO: 7 - DNA, 25 bp, Artificial Sequence
  oligonucleotide LM25 from ARK Scientific, Darmstadt, starting material for preparing the linker ML2025
gatctcgcgagacttagcatgtgac

SEQ ID NO: 8 - DNA, 17 bp, Artificial Sequence
  oligonucleotide designated M13, from ARK Scientific, Darmstadt
caggaaacagcgatgac

SEQ ID NO: 9 - DNA, 33 bp, Artificial Sequence
  Hairpin sequencing primer
tcttcgaatgcactgagcgcattcgaagagatc

SEQ ID NO: 10 - DNA, 19 bp, Artificial Sequence
  oligonucleotide designated T7 from ARK Scientific, Darmstadt
taatacgactcactatagg